(19) United States
(12) Patent Application Publication
(10) Pub. No.: US 2016/0128950 A1
(43) Pub. Date: May 12, 2016

(54) TAPE HAVING TRANSDERMAL ANALGESIC PROPERTIES

(71) Applicant: Sumair MITROO, Fort Lauderdale, FL (US)

(72) Inventor: Sumair Mitroo, Fort Lauderdale, FL (US)

(21) Appl. No.: 14/899,390

(22) PCT Filed: Jun. 27, 2014
(86) PCT No.: PCT/US14/44648

§ 371 Date: Dec. 17, 2015

Related U.S. Application Data
(60) Provisional application No. 61/840,903, filed on Jun. 28, 2013.

(51) Int. Cl. A61K 9/70; A61K 36/185; A61K 31/045; A61K 31/737; A61K 36/9068; A61K 36/714; A61K 36/752; A61K 36/638; A61K 36/79; A61K 36/45; A61K 36/8884; A61K 36/22; A61K 36/48; A61K 36/328; A61F 13/02; A61K 36/232

(52) U.S. Cl. CPC A61K 9/7076; A61K 36/232; A61K 36/185; A61K 31/045; A61K 31/737; A61K 36/9068; A61K 36/714; A61K 36/752; A61K 36/638; A61K 36/79; A61K 36/45; A61K 36/8884; A61K 36/22; A61K 36/48; A61K 36/328; A61K 9/7038; A61F 13/02; A61F 13/0259; A61F 13/0253; A61F 13/0223

(57) ABSTRACT

An adhesive body tape (100) configured for application to skin including a first layer (105), wherein the first layer (105) is for receiving at least one additional layer. The tape further includes a second layer (110) deposited onto the first layer (105). The second layer (110) comprises an adhesive, a first set of ingredients and a second set of ingredients having analgesic properties, and wherein the tape (100) maintains a body part of an individual (710) in proper alignment and wherein the tape (100) transdermally delivers relief of pain to an individual (710).

### CROSS-REFERENCE TO RELATED APPLICATIONS

This patent application claims priority to U.S. provisional patent application No. 61/840,903 filed Jun. 28, 2013 entitled “Kinesiology tape with transdermal analgesic properties.”

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The technical field relates generally to elastic tapes, and more specifically to tape that is applied to an individual's skin.

### BACKGROUND

In 1870, the Japanese Chiropractor Dr. Kenzo Kase developed a highly elastic, breathable and water resistant plaster or tape. Therapeutic tape similar to the tape Dr. Kenzo Kase developed was used for treating athletic injuries and a variety of physical disorders. The tape became useful in therapy to reduce soreness in overused and injured muscles and to accelerate recovery during rehabilitation. Before applying the tape to an individual's skin, an individual could apply forces to stretch the tape to a form or state greater than its original form and then press onto an individual's skin. After being pressed onto an individual's skin in a stretched state, and because of the tape's resilient or elastic properties, the tape would tend to return its original state or form. The elastic properties would cause forces to act on the skin that would have a lifting effect on the skin. This elastic force would compensate for weakened or strained tendons or ligaments underneath the skin and would allow for more ease in movement. This lifting effect on the skin may reduce swelling and inflammation by improving circulation and reducing pain by taking pressure off pain receptors.

In the past, such tape has also been used to restrict an individual's body movement. The tape may assist in maintaining an individual's body in proper anatomical alignment by allowing some movement, but at the same time, restricting some movement when the body part that the tape is pressed against moves.

Pain can effect an athlete's ability to perform. In today's society, because of the amount of money athletes earn, athletes are required to continue playing with pain. Additionally, because people are now conscious of their body weight and appearance, many people choose to continue exercising despite having minor aches and pains in certain areas of their body. Furthermore, it can be cost prohibitive to take oral pain medication in addition to using tape to maintain a body part in the proper anatomical position.

Therefore, a need exists for improvements over the prior art, and more particularly for methods and a device that more effectively reduces pain. There is also a need for reducing the cost of providing pain relief and maintaining an individual's body part in proper anatomical position.

### SUMMARY

A device and method to reduce pain by combining an elastic kinesiology tape to maintain proper kinesthetic alignment and a topical analgesic herbal medicine to further reduce pain is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, the invention is an adhesive body tape configured for application to the human skin comprising a first layer for receiving at least one additional layer. The tape also includes a second layer deposited onto the first layer. The second layer comprises an adhesive, a first set of ingredients and a second set of ingredients. The second layer is configured to have transdermally delivered analgesic properties.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings. Additionally, the left most digit of a reference number identifies the drawing in which the reference number first appears. The accompanying drawings, which are incorporated in and constitute a part of this disclosure, illustrate various example embodiments. In the drawings:

FIG. 1 is an illustration of a perspective view of the tape, in accordance with one embodiment of the present invention;

FIG. 2 is an illustration of a perspective view of the tape, in accordance with another embodiment of the present invention;

FIG. 3 is an illustration of a perspective view of the tape, in accordance with another embodiment of the present invention;

FIG. 4 is an illustration of a perspective view of the tape, in accordance with another embodiment of the present invention;

FIG. 5 is a cross-sectional side view of the tape, in accordance with another embodiment of the present invention;

FIG. 6 is an illustration of a perspective view of the tape rolled up on a tubular body, in accordance with another embodiment of the present invention; and

FIG. 7 is an illustration of a perspective view of the tape applied to an individual, in accordance with another embodiment of the present invention.

### DETAILED DESCRIPTION

The following detailed description refers to the accompanying drawings. Wherever possible, the same reference numbers are used in the drawings and the following description to refer to the same or similar elements. While embodiments of the invention may be described, modifications, adaptations, and other implementations are possible. For example, substitutions, additions, or modifications may be made to the elements illustrated in the drawings, and the methods and devices described herein may be modified by substituting, reordering, or adding stages or elements to the disclosed methods or devices. Accordingly, the following detailed description does not limit the invention. Instead, the proper scope of the invention is defined by the appended claims.

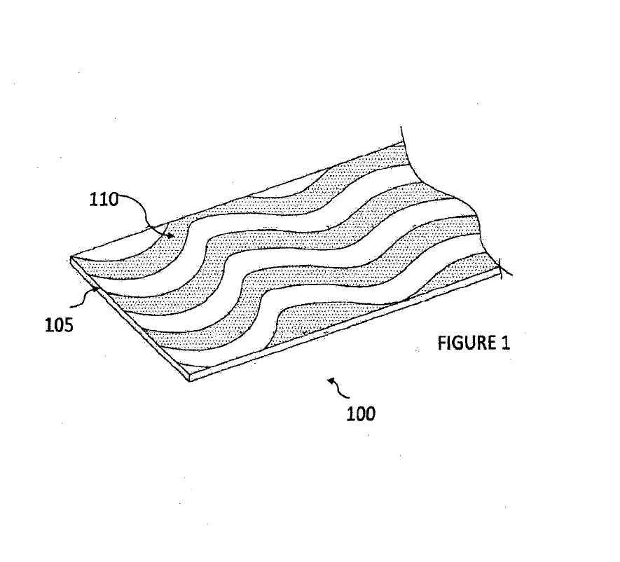

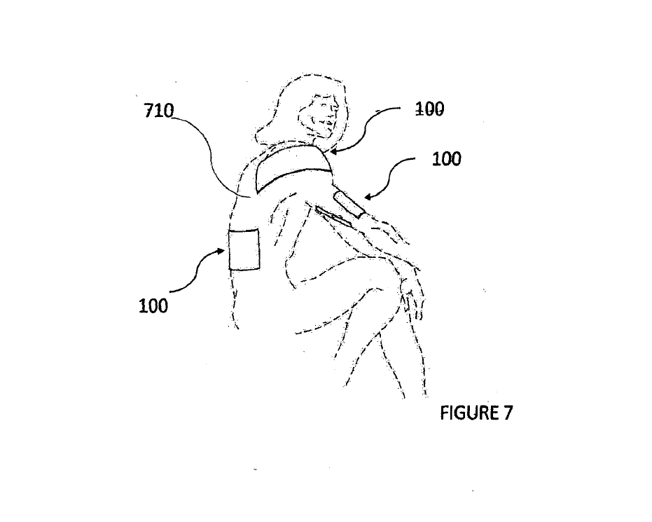

FIG. 1 is perspective view of a portion of the tape 100 according to one embodiment. As illustrated in FIG. 7, the tape 100 is adapted for application to skin of an individual. The tape 100 has a first layer 105 that is adapted for receiving at least one additional layer. The first layer can be made of a fabric (not shown) comprising a group of fibers (not shown) woven together having a certain elasticity. The fibers may be woven together at right angles to one another. In another embodiment, the fibers may have various angles relative to each other. Cotton fibers can be used. The first layer 105 can be either permeable or occlusive. In other embodiments, the fibers may be other materials commonly used for bandages such as nonwovens, cellulose, polyester, polyamide, acetate and blended fabrics or the like. In yet another embodiment, fibers of different materials may be woven together to create the fabric. The woven fabric may be formed of a variety of colors. The fabric may be dyed or may be colored by any other suitable manufacturing process known to those skilled in the art. In some embodiments, the fibers may be melted, frozen or otherwise sealed along the edges to prevent fraying of the first layer 105.

The first layer 105 defines a top surface, a bottom surface, a width and a length. The first layer has a certain elastic or resilient property and can be stretched in all directions. Stated differently, the fabric's length or width can stretch without tearing and substantially return to its original state or form after being stretched. In some embodiments, the fabric layer may have an elasticity of between about 120% and about 140%. In other words, if the fabric is pulled, or when forces are applied along the plane of the surface of the fabric, the length and width of the fabric can be increased and when such force is removed, the fabric then substantially returns to its original shape or form. The top surface of the first layer 105 faces away from or is distal to an individual's skin when the tape is applied to an individual's skin. Opposed to the top surface of the first layer is the bottom surface. The bottom surface, or skin side, of the first layer faces the skin of an individual when the tape is applied to the skin.

A second layer 110 is deposited onto the first layer. The second layer is configured to deliver analgesics transdermally into the skin of an individual. The second layer defines a top surface opposing a bottom surface. The top surface faces away from or is distal to an individual's skin when the tape is applied to an individual's skin. The bottom surface of the second layer is in contact with and adheres to the bottom surface of the first layer. The second layer 110 comprises an adhesive, a first set of ingredients, a second set of ingredients and are configured to have analgesic properties. The first set of ingredients can be active ingredients. The second set of ingredients can be inactive ingredients. The first and second sets of ingredients can be dispersed into the second layer 110 such that the first and second sets of ingredients and adhesive, or adhesive resin, is homogeneous in nature. In other embodiments, the first and second sets of ingredients may be dispersed non-homogeneously throughout the second layer.

In one embodiment, the adhesive can be a water-based acrylic glue. In another embodiment, the adhesive can be a petroleum based glue, including components such as mineral oil, petrolatum, rosin, silicone, and zinc oxide. The adhesive material is an elastic pressure sensitive adhesive that is suitable for long-term skin contact and should be physically and chemically compatible with the first and second sets of ingredients or other additives that are present.

However other pressure sensitive adhesive components, such as polyethyelenes, polysiloxanes, ployisobutylenes, polyacrylates, polyacryclamides, polyurathenes, plasticized ethylene-vinly acetate copolymers and tack rubber may be used. It should also be noted that the material used for the first layer 105 should be incapable of absorbing substantial amounts of the components of the second layer.

In one embodiment, the first set of ingredients includes at least one of the ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidumEucalyptus Oil, Lavender Oil, root bark of bodiner, rhizome arisaema, myrrh, mastic, lanolin, and kirin dried.

In one embodiment, the second set of ingredients includes at least one of the ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried. The second layer is configured to have analgesic properties.

In one embodiment, to form the second layer, the adhesive, first and second sets of ingredients are blended together in order to form one homogeneous mass. For example, this can be accomplished by first combining the adhesive, the first and second sets of ingredients to form a mixture. In one embodiment, the adhesive can be approximately 50% of the entire mixture. However, in other embodiments, the adhesive can be less than or greater than 50% of the entire mixture. Of such entire mixture, the first set of ingredients can comprise approximately 2%-5% of camphor and 1%-4% of menthol. In other embodiments, other ratios and percentages of ingredients may be used. The second set of ingredients can comprise angelica dahurica root, atractylodes lancea root, borneol, Chinese angelica, chondroitin sulfate, ginger, kaempferia galanga root, kusnezoff monkshoot root (raw), kusnezoff monkshoot root, lemon, ligusticum sinenese, chuanxiong root, mineral oil, musk, petrolatum, radix aconiti, rosin, silicone, star anise, wintergreen oil, zinc oxide. However, other ratios of adhesive to analgesic ingredients can be used.

While varying ways of manufacturing the tape are understood, in one embodiment, adhesive, the first and second sets of ingredients can be combined together by a high-speed stirring machine, wherein the mixing and stirring machine causes the mixture to form a mass. After the mixing and stirring process has been completed, the mass can be inserted into a pressing machine in order to deposit the mixture onto the first layer 105. In one embodiment, a pressing machine can deposit or press the mixture onto a large sheet of fabric (such large sheets of fabric are larger sizes of the first layer 105 before being cut to the desired dimension). In one embodiment, the pressing machine can be a heat pressing machine. The process of pressing or depositing the mixture onto the sheets of fabric can be executed at 120° C. However, other temperatures or methods of depositing or applying the adhesive second layer onto the first layer can be used.

After the mass has been deposited onto the surface of the large sheet of fabric, a 24-hour cooling period is required before cutting the sheets into a desired shape and size. In one embodiment, the desired size of the tape is a 5 cm wide by 5 m long. Other dimensions for the length and width can be used depending on the body part to which the tape 100 is to be applied or as desired for various applications contemplated by the individual.

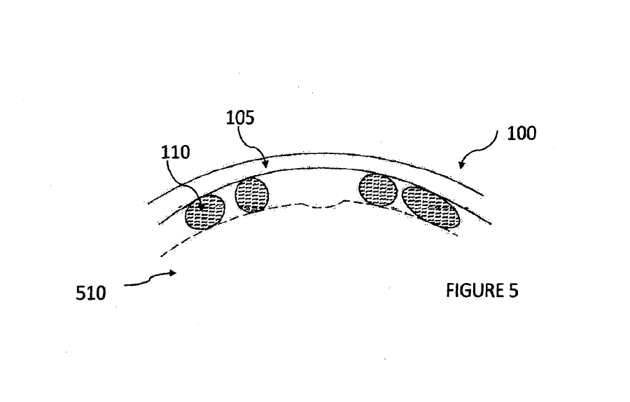

The top surface or skin side of the second layer 105 is formed to adhere the tape to a human's skin and is proximal to and comes into contact with an individual's skin when the tape is pressed onto an individual's skin (as shown in FIG. 5). In one embodiment, the adhesive comprises a water acrylic based adhesive. In other embodiments, other adhesives used for bonding or fusing objects or materials together may be used. The amount of adhesive used in the second layer may vary on the use. If a stronger adhesive hold is required, then an adhesive having stronger adhesive properties may be used. Alternatively, the percentage of the second layer comprised of adhesive may be increased. Or in another alternative, the amount of second layer deposited onto the first layer may be increased.

In one embodiment, the second layer is coupled or attached to the first layer by an adhesive bond. However, the bottom surface of the first layer and the bottom surface of the second layer may be coupled or attached using a chemical, mechanical, thermal, electromagnetic or by any method that is suitable for bonding fabrics to other materials.

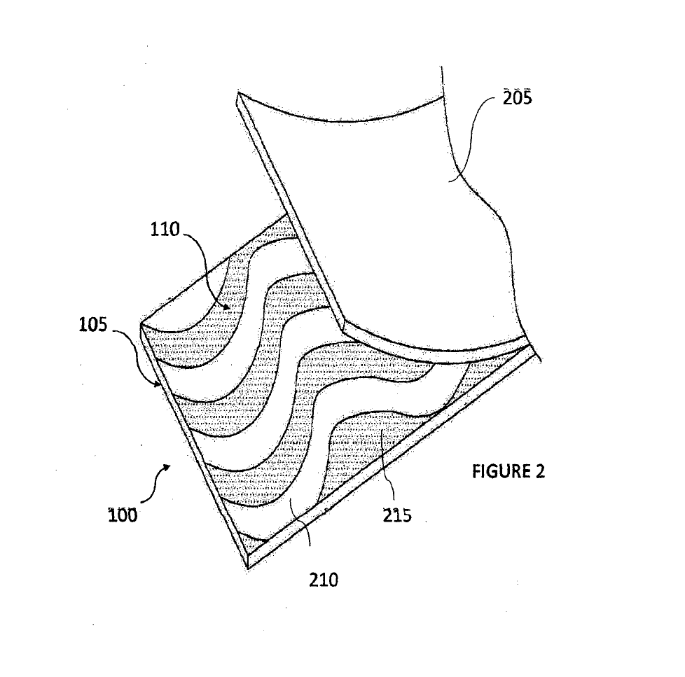

FIG. 2 is another perspective view of one embodiment of the present invention. In this embodiment, the tape 100 also includes a removably attached film or backing 205 used to cover or protect the adhesive layers during manufacturing, shipping, storage, etc. In FIG. 2, the backing 205 is partially removed. The backing may be made of material, such as wax paper or other materials used to protect adhesive materials. The backing 205 is present during storage and prior to application onto the skin and is adapted to protect or cover the entire second layer. Immediately prior to use, the backing is removed from the tape 100 so that the tape may be adhered to the skin.

As shown in FIGS. 1 and 2, the second layer does not cover the entire surface of first layer resulting in uncovered 210 and covered sections 205 of the bottom surface of the first layer. These uncovered and covered sections 210, 215 define a pattern. As shown in FIG. 2, the pattern may define a wave like pattern, elongated curved bands or features. Other patterns may also be defined including chevrons, squares, or other patterns defining various geometrical shapes. In FIG. 2, the second layer covers approximately fifty percent (50%) of the first layer. In another embodiment (not shown), the second layer may cover the entire surface of the first layer. The amount of covered portions may be increased to increase the adhesive hold and/or to increase the amount of analgesics to be delivered.

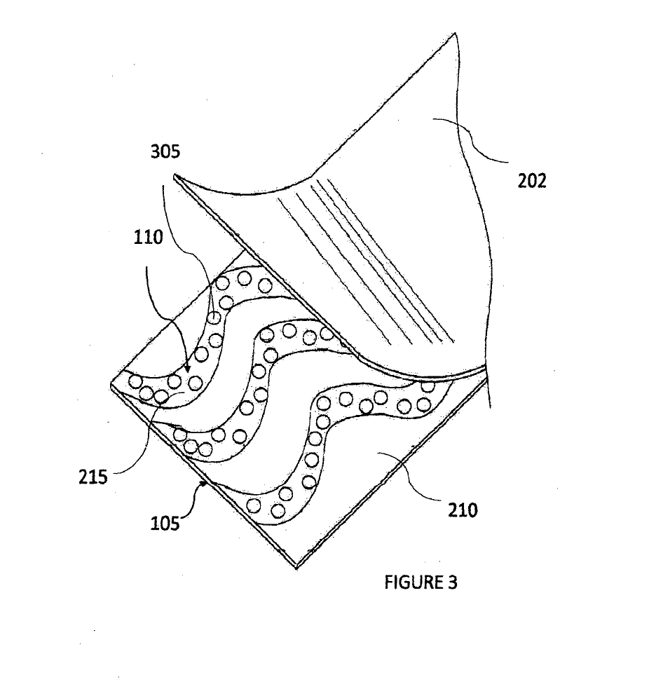

FIG. 3 is a perspective view of another embodiment of the present invention. FIG. 3 illustrates another ratio of covered portions 215 to uncovered portions. Additionally, in this embodiment there are more uncovered portions 210 than covered portions 215. Other ratios of covered portions to uncovered portions may also be used. The various ratios of uncovered portions to covered portions can form patterns that promote the passage of air or moisture on an individual's skin that can also increase the amount of time an individual can wear the tape. The patterns may be formed by various cutting dies. However, other means of creating the patterns known to those skilled in the art can be used.

FIG. 3 also illustrates that additional layers can be coupled to, attached or deposited on top of the first and second layers. The additional layer may cover portions of the second layer 110 defining a pattern. In this embodiment, the additional layer defines a group of bodies 305. The bodies are substantially circular shaped. However, other patterns or designs may be used.

Each additional layer may comprise an adhesive and at least two of the ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried. In one embodiment, these additional layers may be coupled or attached to the first and second layers by an adhesive bond. However, other means of coupling or attaching the additional layers to the other layers may be utilized including a chemical, mechanical, thermal, electromagnetic or by any method that is suitable for bonding fabrics.

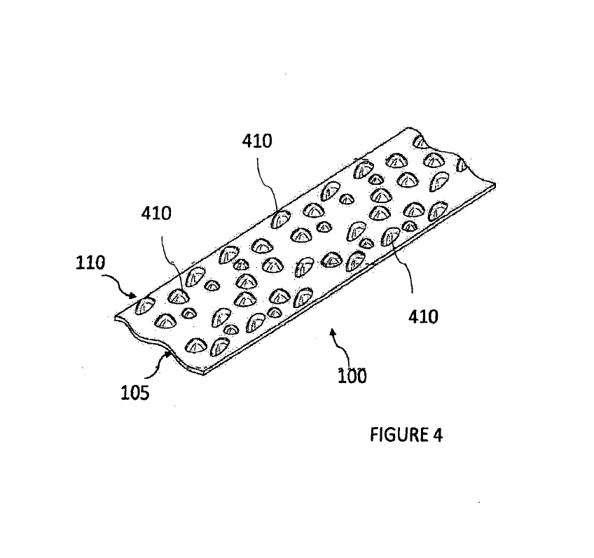

FIG. 4 is a perspective view of one embodiment of the present invention. FIG. 4 illustrates a portion of tape 100 having a plurality of bodies 410 forming the second layer 110 deposited onto the first layer 105. As in FIGS. 1-3, the bodies forming the second layer 110 include an adhesive, a first set of ingredients and a second set of ingredients and is configured to have analgesic properties. The first and second sets of ingredients can comprise the same ingredients as described above in FIGS. 1 and 2. The first set of ingredients can be active ingredients. The second set of ingredients can be inactive ingredients.

FIG. 5 is a cross-sectional side view of a portion of the tape 100 applied to the skin 510 of an individual. FIG. 5 illustrates a second layer 110 adhered or adhesively coupled to the skin of an individual. In this particular embodiment, the second layer comprises substantially circular shaped bodies deposited on the bottom surface of the first layer 105.

In one non-limiting embodiment, and as illustrated in FIG. 1, the tape has a planer elongated rectangular shape. The first layer 105 is adapted for receiving a second layer. The first layer is made of fabric comprising a group of cotton or polymeric fibers woven together having a certain elasticity. The fibers are woven together at substantially right angles to one another. The first layer has a top surface and a bottom surface. A second layer 110 is deposited onto the bottom surface of the first layer 105. The second layer can be deposited onto the first layer in the manner described above. The second layer 110 comprises an adhesive, a first set of ingredients, a second set of ingredients and is configured to have analgesic properties. The first and second sets of ingredients is dispersed into the second layer such that the first and second sets of ingredients and adhesive are homogeneous in nature. In this non-limiting embodiment, the adhesive may be a petroleum based glue. The first set of ingredients comprises Camphor and Menthol. The first set of ingredients are active ingredients. The adhesive comprises approximately fifty percent (50%) of the second layer. The adhesive may comprise lanolin, mineral oil, petrolatum, rosin, silicone, and zinc oxide. Camphor comprises approximately 2-5% of the total sum (or combined total) of the first and second sets of ingredients and menthol comprises 1-4% of the total sum of the first and second sets of ingredients. In another embodiment, camphor comprises approximately 3% of the total sum of the first and second sets of ingredients and menthol comprises approximately 2.52% of the total sum of the first and second sets of ingredients. In yet another embodiment, camphor comprises approximately 3% of the second layer (which includes the adhesive and the first and second sets of ingredients) and menthol comprises approximately 2.52% of the second layer. In yet another embodiment, camphor comprises approximately 2-5% and menthol comprises approximately 1-4% of the second layer. The second set of ingredients are angelica, lancea root, borneol, chondroitin sulfate, ginger, kusnezoff monkshoot root (raw), lemon, ligusticum sinenese subsp chuanxiong root, radix aconiti, star anise, wintergreen oil, and rhizome arisaema, mastic, root bark of bodiner, kirin dried, and myrrh. The second set of ingredients are inactive ingredients.

In this non-limiting embodiment, the second layer does not cover the entire surface of the first layer resulting in uncovered 210 and covered sections 205 of the bottom surface of the first layer. These uncovered and covered sections 210, 215 can define a pattern. As shown in FIG. 2, the pattern defines a plurality of elongated curved bands or wave like features. The second layer covers approximately fifty percent (50%) of the first layer. As shown in FIG. 2, a removably attached backing or liner 205 protects the second layer during manufacturing, shipping, storage, etc. As shown in FIG. 2, the backing 205 is partially removed. In this embodiment, the backing may be made of wax paper or the like. In this embodiment, the backing is removed before applying the tape to the skin.

Before applying the tape onto an individual, forces may be applied along the plane of the tape causing the fibers of the first layer to stretch greater than its initial state or form. While the first layer is in a stretched state, the skin side of the second layer 110 is pressed onto the skin 510. Upon doing this, the adhesive of the second layer adheres the tape to an individual's skin. The elastic or resilient properties of the first layer act on the stretched fibers of the first layer to return the first layer substantially to its initial form or state. As the tape returns substantially to its initial form or state, forces act upon the individual's skin thereby lifting the individual's skin. This lifting effect may reduce swelling and inflammation by improving circulation and reducing pain by taking pressure off pain receptors.

In one embodiment, the area of skin 510 is cleaned before the tape is applied to it. An individual or user could clean the skin using water, water and soap, rubbing alcohol, or any other method or device to remove dirt and other external substances from the skin. The backing 205 is removed by peeling the backing 205 from the top of the second layer 110.

The first layer also assists to maintain an individual's body in proper anatomical alignment by allowing some movement but at the same time restricting movement when the body part that the tape is pressed against moves. The forces acting on the skin as a result of the tape's placement onto the skin may assist in maintaining an individual's body in proper anatomical alignment by allowing some movement but at the same time restricting movement. The elastic properties also cause forces to act on the skin and have a lifting effect on the skin. This lifting effect on the skin may reduce swelling and inflammation by improving circulation and reducing pain by taking pressure off pain receptors.

The second layer 110 is configured to deliver the analgesic components within the second layer through the skin providing temporary pain relief of minor aches and pains of muscles and joints associated with backache, arthritis, strains, bruises and strains. In one embodiment, the tape can provide pain relief to an individual for 12 hours. In some embodiments, if the tape is removed, the tape will continue to provide pain relief for up to half an hour after removal of the tape.

After the tape is applied to the skin, portions of the first and second sets of ingredients are absorbed by the skin and are diffused into skin and surrounding tissue. The first and second sets of ingredients are pain receptor blockers. After being absorbed into skin and the surrounding tissue, the ingredients activate certain receptors, such as NMDA or kappa opioid receptors, providing natural pain relief to the surrounding skin and tissue.

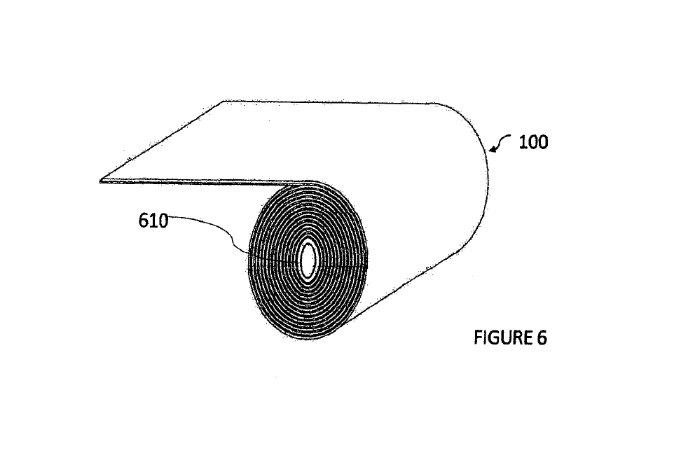

As mentioned above, the tape may be stored in various ways. For example, the tape may be cut into individual sections for various uses. In one embodiment, as shown in FIG. 6, the tape may be rolled up on a tubular or cylindrical shaped body or mandrel 610 and may be provided as a roll from which strips may be cut or torn to a desired size and subsequently applied in a desired pattern, or alternately, provided in pre-sized, pre-shaped and/or pre-configured shapes or sizes for use in specified applications.

FIG. 7 illustrates a perspective view of an individual 710 having the tape 100 applied to the skin. As mentioned above, the tape can be configured and sized for various body parts. For instance, the elbow or wrist may require a different size or length tape than the thigh or hamstring.

The tape may also be provided as part of a kit for providing pain relief. The kit can include the tape 100 as well as a cleaning element to clean the skin where the tape is to be applied. A cleaning element, such as a cloth, may contain solvents to remove water-soluble and/or lipid—solvable debris and dirt from the skin.

While certain embodiments have been described and shown in the accompanying drawings, such embodiments are merely illustrative and are not restrictive of the scope of the disclosure, and this disclosure is not limited to the specific instructions and arrangement shown and described, since various other additions and modifications to and deletions from, the described embodiments will be apparent to one of ordinary skill in the art. Thus, the scope of the disclosure is only limited by the literal language, and equivalents, of the claims which follow.

What I claim is:

1. An adhesive body tape configured for application to skin comprising:
a first layer, wherein the first layer is for receiving at least one additional layer; and
a second layer deposited onto the first layer, wherein the second layer comprises an adhesive, a first set of ingredients and a second set of ingredients;
wherein the first set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried;
wherein the second set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried; and,
wherein said second layer is configured to have at least one analgesic property.

2. The adhesive body tape of claim 1, wherein the tape further comprises a removably attached backing covering the second layer.

3. The adhesive body tape of claim 1, wherein the adhesive comprises approximately fifty percent (50%) of the second layer.

4. The adhesive body tape of claim 1, wherein the adhesive is a water based acrylic glue.

5. The adhesive body tape of claim 1, wherein the first layer comprises a group of fibers woven together.

6. The adhesive body tape of claim 1, wherein the first layer is occlusive or permeable.

7. The adhesive body tape of claim 1, wherein the second layer does not cover the entire first layer defining a pattern.

8. The adhesive body tape of claim 7, wherein the pattern is a plurality of elongated curved bands.

9. The adhesive body tape of claim 1, wherein the first layer further comprises at least one elastic property, wherein the tape maintains a body part of an individual in proper anatomical alignment, and wherein the tape transdermally delivers temporary relief of pain to an individual.

10. The adhesive body tape of claim 2, wherein the backing is removed prior to applying the tape to skin.

11. A method of maintaining a body part of an individual in proper alignment and providing relief of pain comprising:
a. applying an adhesive body tape configured for application to skin, wherein the tape comprises:
a first layer, wherein the first layer is for receiving at least one additional layer; and
a second layer, wherein the second layer is deposited onto first layer, wherein the second layer comprises an adhesive, a first set of ingredients and a second set of ingredients,
wherein the first set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried,
wherein the second set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried, and
wherein second layer is configured to have at least one analgesic property;
b. wearing the tape, wherein wearing the tape provides relief of pain for the individual for up to twelve hours; and,
c. removing the tape, wherein the first and second sets of ingredients continue providing relief from pain for approximately one half (½) hour after removing the tape.

12. The method of claim 11, wherein the tape further comprises a removably attached backing covering the second layer, and wherein the method further comprises removing the backing prior to applying to an area of skin.

13. The method of claim 12, wherein the method further comprises cleaning the skin prior to applying the tape.

14. The method of claim 13, wherein the first layer further comprises at least one elastic property, and wherein the method further comprises (a) stretching the tape before applying the tape on to the skin, and (b) applying the tape onto skin in a stretched state.

15. An adhesive body tape configured for application to skin comprising:
a first layer comprising a fabric having a group of fibers woven together, wherein the first layer comprises at least one elastic property, and wherein the first layer is for receiving at least one additional layer;
a second layer deposited onto the first layer, wherein the second layer comprises an adhesive, a first set of ingredients and a second set of ingredients;
wherein the first set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried;
wherein the second set of ingredients includes at least one or more ingredients derived from various roots of plants, oils, minerals, fruits and herbs, such as camphor, menthol, angelica dahurica root, atractylodes lancea root, borneol, Chinese angelica, chondroitin sulfate, ginger, kaempferia galanga root, kusnezoff monkshoot root (raw), kusnezoff monkshoot root, lemon, lime, ligusticum sinenese/subsp. chuanxiong root, mineral oil, musk, petrolatum, radix aconiti, rosin, silicone, star anise, wintergreen oil, zinc oxide, Camellia tea, Sargentgloryvine Stem, Radix Notoginseng, Obtuseleaf Erycibe Stem, Aconite Root, Nux Vomica, Angelica Dahurica, Rhizoma, Corydalis, Safflower, Draconis Resin, Frankincense, Myrrh, Caulis Spatholobi, Manchurian Wildginger, Radix Salviae Miltiorrhizae, Fortune's Drynaria Rhizome, Radix Cynanchi Paniculati, Safflower, Rhubarb, Weeping Forsythia Seed Pod, Turmeric, Tangerine Peel, Red Peony Root, Fortune's Drynaria Rhizome, Cape Jasmine Fruit, White Peony Root, Zedoary, Rehmannia Root (raw), Slenderstyle Acanthopanax Bark, Tree Peony Bark, Ethyl, Alcohol, Peppermint Oil, Pinellia Tuber, Zanthoxylum nitidum, Eucalyptus Oil, Lavender Oil, root bark of bodiner, rhizome arisaema, myrrh, mastic, lanolin, and kirin dried;
wherein the second layer is configured to have at least one analgesic property, and wherein the adhesive comprises approximately fifty percent (50%) of the second layer; and,
a removably attached backing covering the second layer.

16. The adhesive body tape of claim 15, wherein the adhesive is a water based acrylic glue.

17. The adhesive body tape of claim 16, wherein the first layer is occlusive or permeable.

18. The adhesive body tape of claim 17, wherein the second layer does not cover the entire first layer defining a pattern.

19. The adhesive body tape of claim 18, wherein the pattern is a plurality of elongated curved bands.

20. The adhesive body tape of claim 19, wherein the tape maintains a body part of an individual in proper alignment and wherein the tape transdermally delivers relief of pain to an individual.

21. An adhesive body tape configured for application to skin comprising:
a first layer, wherein the first layer is for receiving a second layer; and
a second layer deposited onto the first layer, wherein the second layer comprises an adhesive, a first set of ingredients and a second set of ingredients;
wherein the first set of ingredients are camphor and menthol;
wherein the second set of ingredients are angelica, lancea root, borneol, chondroitin sulfate, ginger, kusnezoff monkshoot root (raw), lemon, ligusticum sinenese subsp chuanxiong root, radix aconiti, star anise, wintergreen oil, rhizome arisaema, mastic, root bark of bodiner, kirin dried, and myrrh; and
wherein said second layer is configured to have at least one analgesic property.

22. The adhesive body tape of claim 21, wherein the tape further comprises a removably attached backing covering the second layer.

23. The adhesive body tape of claim 21, wherein the adhesive comprises approximately fifty percent (50%) of the second layer.

24. The adhesive body tape of claim 21, wherein camphor comprises approximately 2-5% of the total sum of the first and second sets of ingredients.

25. The adhesive body tape of claim 21, wherein menthol comprises approximately 1-4% of the total sum of the first and second sets of ingredients.

26. The adhesive body tape of claim 21, wherein camphor comprises approximately 3% of the total sum of the first and second sets of ingredients.

27. The adhesive body tape of claim 26, wherein menthol comprises approximately 2.52% of the total sum of the first and second sets of ingredients.

28. The adhesive body tape of claim 21, wherein camphor comprises approximately 3% of the second layer.

29. The adhesive body tape of claim 28, wherein menthol comprises approximately 2.52% of the second layer.

30. The adhesive body tape of claim 21, wherein camphor comprises approximately 2-5% of the second layer.

31. The adhesive body tape of claim 30, wherein menthol comprises approximately 1-4% of the second layer.

32. The adhesive body tape of claim 21, wherein the adhesive is a petroleum based glue.

33. The adhesive body tape of claim 21, wherein the first layer comprises a group of fibers woven together.

34. The adhesive body tape of claim 33, wherein the fibers are cotton.

35. The adhesive body tape of claim 21, wherein the second layer does not cover the entire first layer defining a pattern.

36. The adhesive body tape of claim 35, wherein the pattern is a plurality of elongated curved bands.

37. The adhesive body tape of claim 21, wherein the first layer further comprises a least one elastic property, wherein the tape maintains a body part of an individual in proper anatomical alignment, and wherein the tape transdermally delivers temporary relief of pain to an individual.

38. The adhesive body tape of claim 22, wherein the backing is removed prior to applying the tape to skin.

39. The adhesive body tape of claim 32, wherein the glue comprises mineral oil, petrolatum, rosin, silicone, and zinc oxide.
